(19) United States
(12) Patent Application Publication
(10) Pub. No.: US 2026/0303973 A1
(43) Pub. Date: Oct. 1, 2026

(54) SYSTEMS AND METHODS FOR IMPLEMENTING A ZOOM FEATURE ASSOCIATED WITH AN IMAGING DEVICE IN AN IMAGING SPACE

(71) Applicant: Intuitive Surgical Operations, Inc., Sunnyvale, CA (US)

(72) Inventors: Florian Wirth, Lahnau (DE); Peter Liebetraut, Gundelfingen (DE); Rohitkumar Godhani, Milpitas, CA (US)

(21) Appl. No.: 19/479,628

(22) PCT Filed: Jul. 17, 2024
(86) PCT No.: PCT/US2024/038392

§ 371 Date: Oct. 28, 2025

Related U.S. Application Data
(60) Provisional application No. 63/527,430, filed on Jul. 18, 2023.

(51) Int. Cl. H04N 23/69; A61B 34/37; G06T 7/55; H04N 23/62; H04N 23/695

(52) U.S. Cl. CPC H04N 23/69; A61B 34/37; G06T 7/55; H04N 23/62; H04N 23/695; G06T 2207/30004; G06T 2207/30244

(57) ABSTRACT

An illustrative system includes a memory storing instructions and a processor communicatively coupled to the memory. The processor may be configured to execute the instructions to perform a process comprising receiving an instruction to move an imaging device, which is located within an imaging space and coupled to a robotic manipulating arm of a computer-assisted surgical system, towards an object in the imaging space, determining, based on the instruction, that the imaging device is located at a position that is at or near a threshold distance from the object in the imaging space, preventing the imaging device from moving closer to the object than the threshold distance, and automatically activating, in response to preventing the imaging device from moving closer to the object than the threshold distance, an image zoom feature to zoom in a view of the imaging space.

### RELATED APPLICATIONS

This application claims priority to U.S. Provisional Patent Application No. 63/527,430, filed Jul. 18, 2023, the contents of which is hereby incorporated by reference in its entirety.

### BACKGROUND INFORMATION

A computer-assisted surgical system that employs robotic and/or teleoperation technology typically includes a stereoscopic image viewer configured to provide, for display to a surgeon, images of an imaging space (e.g., a surgical space) as captured by an imaging device such as an endoscope. While the surgeon's eyes are positioned in front of viewing lenses of the stereoscopic image viewer, the surgeon may view the images of the surgical space while remotely manipulating one or more surgical instruments located within the surgical space. The surgical instruments are attached to one or more manipulator arms of a surgical instrument manipulating system included as part of the computer-assisted surgical system.

In addition to remotely manipulating the surgical instruments, the surgeon may remotely manipulate the imaging device, which is attached to one of the manipulating arms, to change the position and/or view of the imaging space captured by the imaging device. For example, the surgeon may move the imaging device closer to an object within the imaging space to get a closer view of the object. However, moving the imaging device too close to the object may cause damage to the object and/or result in reduced image quality of images captured by the imaging device. For example, moving the imaging device too close to tissue within the imaging space may result in burning of the tissue, fogging of the imaging device, and/or splatter on the imaging device, which may require removal of the imaging device for cleaning or replacement. It may be possible for a surgeon to manually activate/deactivate a zoom function of an imaging device during a procedure to zoom in on an object and possibly avoid such negative effects. However, such manual activation/deactivation of a zoom function undesirably increases the complexity of a procedure and results in additional workload for the surgeon. Accordingly, there remains room to improve the implementation of a zoom function of an imaging device used in an imaging space.

### SUMMARY

An example system comprises a memory storing instructions; and a processor communicatively coupled to the memory and configured to execute the instructions to perform a process comprising receiving an instruction to move an imaging device, which is located within an imaging space and coupled to a robotic manipulating arm of a computer-assisted surgical system, towards an object in the imaging space, determining, based on the instruction, that the imaging device is located at a position that is at or near a threshold distance from the object in the imaging space, preventing the imaging device from moving closer to the object than the threshold distance, and automatically activating, in response to the preventing of the imaging device from moving closer to the object than the threshold distance, an image zoom feature to zoom in a view of the imaging space.

An example computer program product embodied in a non-transitory computer readable storage medium comprises computer instructions for performing a process comprising receiving an instruction to move an imaging device, which is located within an imaging space and coupled to a robotic manipulating arm of a computer-assisted surgical system, towards an object in the imaging space, determining, based on the instruction, that the imaging device is located at a position that is at or near a threshold distance from the object in the imaging space, preventing the imaging device from moving closer to the object than the threshold distance, and automatically activating, in response to preventing the imaging device from moving closer to the object than the threshold distance, an image zoom feature to zoom in a view of the imaging space.

An additional example computer program product comprises instructions which, when executed by a computer, cause the computer to perform a process comprising receiving an instruction to move an imaging device, which is located within an imaging space and coupled to a robotic manipulating arm of a computer-assisted surgical system, towards an object in the imaging space, determining, based on the instruction, that the imaging device is located at a position that is at or near a threshold distance from the object in the imaging space, preventing the imaging device from moving closer to the object than the threshold distance, and automatically activating, in response to preventing the imaging device from moving closer to the object than the threshold distance, an image zoom feature to zoom in a view of the imaging space.

An example method comprises receiving, by an automatic zoom system, an instruction to move an imaging device, which is located within an imaging space and coupled to a robotic manipulating arm of a computer-assisted surgical system, towards an object in the imaging space, determining, by the automatic zoom system and based on the instruction, that the imaging device is located at a position that is at or near a threshold distance from the object in the imaging space, preventing, by the automatic zoom system, the imaging device from moving closer to the object than the threshold distance, and automatically activating, by the automatic zoom system and in response to preventing the imaging device from moving closer to the object than the threshold distance, an image zoom feature to zoom in a view of the imaging space.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an example computer-assisted surgical system according to principles described herein.

FIG. 2 illustrates an example automatic zoom system according to principles described herein.

FIGS. 3A and 3B illustrate example views of an imaging space according to principles described herein.

FIG. 4 illustrates an example flow chart depicting various operations that may be performed by the automatic zoom system illustrated in FIG. 2 according to principles described herein.

FIG. 5 illustrates an example view of an imaging space and a virtual pivot point that may be implemented according to principles described herein.

FIG. 6 illustrates an example method for implementing a zoom feature associated with an imaging device in an imaging space according to principles described herein.

FIG. 7 illustrates an example computing device according to principles described herein.

### DETAILED DESCRIPTION

Systems and methods for implementing a zoom feature associated with an imaging device in an imaging space are described herein. As will be described in more detail below, an illustrative system includes a memory that stores instructions and a processor communicatively connected to the memory. The processor is configured to execute the instructions to perform a process comprising receiving an instruction to move an imaging device, which is located within an imaging space and coupled to a robotic manipulating arm of a computer-assisted surgical system, towards an object in the imaging space, determining, based on the instruction, that the imaging device is located at a position that is at or near a threshold distance from the object in the imaging space, preventing the imaging device from moving closer to the object than the threshold distance, and automatically activating, in response to preventing the imaging device from moving closer to the object than the threshold distance, an image zoom feature to zoom in a view of the imaging space.

Various advantages and benefits are associated with systems and methods described herein. For example, systems and methods such as those described herein may facilitate quick and/or convenient implementation of a zoom feature associated with an imaging device. Instead of requiring a user (e.g., a surgeon) to manually activate/deactivate a zoom feature, systems and methods such as those described herein may facilitate activating a zoom feature automatically based on a predefined condition being satisfied. In so doing, systems and methods such as those described herein may simplify procedures performed within the imaging space and/or improve usability of a computer-assisted surgical system. In addition, systems and methods such as those described herein may facilitate minimizing or preventing negative effects (e.g., tissue damage, fogging, splatter, etc.) that may otherwise occur if an imaging device is moved too close to an object (e.g., tissue) in an imaging space. These and other benefits that may be realized by the systems and methods described herein will be evident from the disclosure that follows.

Example systems described herein may be configured to operate as part of or in conjunction with a plurality of different types of computer-assisted surgical systems. The different types of computer-assisted surgical systems may include any type of computer-assisted surgical system as may serve a particular implementation, such as a computer-assisted surgical system designed for use in minimally-invasive medical procedures, for example. In certain examples, a type of computer-assisted surgical system may include a system in which one or more surgical devices (e.g., surgical instruments) are manually (e.g., laparoscopically) controlled by a user. In certain examples, a type of computer-assisted surgical system may include a robotic surgical system configured to facilitate operation one or more smart instruments (e.g., smart sub-surface imaging devices) that may be manually and/or robotically controlled by a user. In certain implementations, the plurality of different types of computer-assisted surgical systems may be of different types at least because they include different types of surgical instrument manipulating systems. For example, a first computer-assisted surgical system may include a first type of surgical instrument manipulating system, a second computer-assisted surgical system may include a second type of surgical instrument manipulating system, and a third computer-assisted surgical system may include a third type of surgical instrument manipulating system.

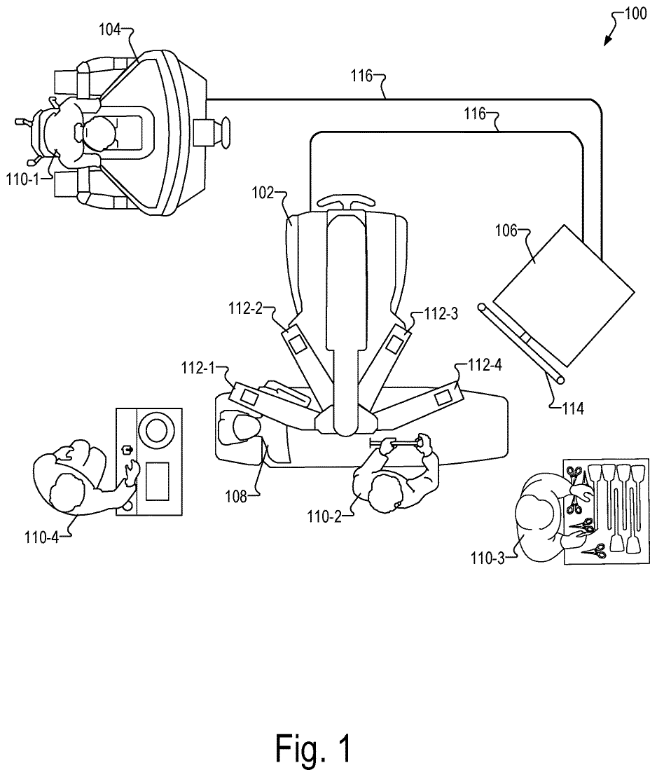

Each type of surgical instrument manipulating system may have a different architecture (e.g., a manipulator arm architecture), have a different kinematic profile, and/or operate according to different configuration parameters. An illustrative computer-assisted surgical system with a first type of surgical instrument manipulating system will now be described with reference to FIG. 1. The described computer-assisted surgical system is illustrative and not limiting. Systems such as those described herein may operate as part of or in conjunction with the described computer-assisted surgical system and/or any other suitable computer-assisted surgical system.

FIG. 1 illustrates an example computer-assisted surgical system 100 (“surgical system 100”). As shown, surgical system 100 may include a surgical instrument manipulating system 102 (“manipulating system 102”), a user control system 104, and an auxiliary system 106 communicatively coupled one to another.

Surgical system 100 may be utilized by a surgical team to perform a computer-assisted surgical procedure on a patient 108. As shown, the surgical team may include a surgeon 110-1, an assistant 110-2, a nurse 110-3, and an anesthesiologist 110-4, all of whom may be collectively referred to as “surgical team members 110.” Additional or alternative surgical team members may be present during a surgical session as may serve a particular implementation.

While FIG. 1 illustrates an ongoing minimally invasive surgical procedure, surgical system 100 may similarly be used to perform open surgical procedures or other types of surgical procedures that may similarly benefit from the accuracy and convenience of surgical system 100. Additionally, it will be understood that the surgical session throughout which surgical system 100 may be employed may not only include an operative phase of a surgical procedure, as is illustrated in FIG. 1, but may also include preoperative, postoperative, and/or other suitable phases of the surgical procedure. A surgical procedure may include any procedure in which manual and/or instrumental techniques (e.g., teleoperated instrumental techniques) are used on a patient to investigate, diagnose, or treat a physical condition of the patient. Additionally, a surgical procedure may include any procedure that is not performed on a live patient, such as a calibration procedure, a simulated training procedure, and an experimental or research procedure.

As shown in FIG. 1, surgical instrument manipulating system 102 may include a plurality of manipulator arms 112 (e.g., manipulator arms 112-1 through 112-4) to which a plurality of robotic surgical instruments (“robotic instruments”) (not shown) may be coupled. As used herein, a “robotic instrument” refers to any instrument that may be directly attached to (e.g., plugged into, fixedly coupled to, mated to, etc.) a manipulator arm (e.g., manipulator arm 112-1) such that movement of the manipulator arm directly causes movement of the instrument. Each robotic instrument may be implemented by any suitable therapeutic instrument (e.g., a tool having tissue-interaction functions), imaging device (e.g., an endoscope), diagnostic instrument, or the like that may be used for a computer-assisted surgical procedure (e.g., by being at least partially inserted into patient 108 and manipulated to perform a computer-assisted surgical procedure on patient 108). In some examples, one or more of the robotic instruments may include force-sensing and/or other sensing capabilities.

In the example shown in FIG. 1, manipulator arms 112 of manipulating system 102 are attached on a distal end of an overhead boom that extends horizontally. However, manipulator arms 112 may have other configurations in certain implementations. In addition, while manipulating system 102 is depicted and described herein as including four manipulator arms 112, it will be recognized that manipulating system 102 may include only a single manipulator arm 112 or any other number of manipulator arms as may serve a particular implementation.

Manipulator arms 112 and/or robotic instruments attached to manipulator arms 112 may include one or more displacement transducers, orientational sensors, and/or positional sensors (hereinafter “surgical system sensors”) used to generate raw (e.g., uncorrected) kinematics information. One or more components of surgical system 100 may be configured to use the kinematics information to track (e.g., determine positions of) and/or control the robotic instruments.

In addition, manipulator arms 112 may each include or otherwise be associated with a plurality of motors that control movement of manipulator arms 112 and/or the surgical instruments attached thereto. For example, manipulator arm 112-1 may include or otherwise be associated with a first internal motor (not explicitly shown) configured to yaw manipulator arm 112-1 about a yaw axis. In like manner, manipulator arm 112-1 may be associated with a second internal motor (not explicitly shown) configured to drive and pitch manipulator arm 112-1 about a pitch axis. Likewise, manipulator arm 112-1 may be associated with a third internal motor (not explicitly shown) configured to slide manipulator arm 112-1 along insertion axis. Manipulator arms 112 may each include a drive train system driven by one or more of these motors in order to control the pivoting of manipulator arms 112 in any manner as may serve a particular implementation. As such, if a robotic instrument attached, for example, to manipulator arm 112-1 is to be mechanically moved, one or more of the motors coupled to the drive train may be energized to move manipulator arm 112-1.

Robotic instruments attached to manipulator arms 112 may each be positioned in an imaging space. An “imaging space” as used herein may refer to any space or location where an imaging operation may be performed by an imaging device such as described herein. In certain examples, an imaging space may correspond to a surgical space. A “surgical space” may, in certain examples, be entirely disposed within a patient and may include an area within the patient at or near where a surgical procedure is planned to be performed, is being performed, or has been performed. For example, for a minimally invasive surgical procedure being performed on tissue internal to a patient, the surgical space may include the tissue, anatomy underlying the tissue, as well as space around the tissue where, for example, robotic instruments and/or other instruments being used to perform the surgical procedure are located. In other examples, a surgical space may be at least partially disposed external to the patient at or near where a surgical procedure is planned to be performed, is being performed, or has been performed on the patient. For instance, surgical system 100 may be used to perform an open surgical procedure such that part of the surgical space (e.g., tissue being operated on) is internal to the patient while another part of the surgical space (e.g., a space around the tissue where one or more instruments may be disposed) is external to the patient. A robotic instrument may be referred to as being positioned or located at or within a surgical space when at least a portion of the robotic instrument (e.g., a distal portion of the robotic instrument) is located within the surgical space. Example imaging spaces and/or images of imaging spaces will be described herein.

User control system 104 may be configured to facilitate control by surgeon 110-1 of manipulator arms 112 and robotic instruments attached to manipulator arms 112. For example, surgeon 110-1 may interact with user control system 104 to remotely move, manipulate, or otherwise teleoperate manipulator arms 112 and the robotic instruments. To this end, user control system 104 may provide surgeon 110-1 with one or more images (e.g., high-definition three-dimensional (3D) images) of a surgical space associated with patient 108 as captured by an imaging device. In certain examples, user control system 104 may include a stereoscopic image viewer having two displays where stereoscopic images (e.g., 3D images) of a surgical space associated with patient 108 and generated by a stereoscopic imaging system may be viewed by surgeon 110-1. Surgeon 110-1 may utilize the images to perform one or more procedures with one or more robotic instruments attached to manipulator arms 112.

To facilitate control of robotic instruments, user control system 104 may include a set of master controls (not shown). These master controls may be manipulated by surgeon 110-1 to control movement of robotic instruments (e.g., by utilizing robotic and/or teleoperation technology). The master controls may be configured to detect a wide variety of hand, wrist, and finger movements by surgeon 110-1. In this manner, surgeon 110-1 may intuitively perform a surgical procedure using one or more robotic instruments.

User control system 104 may further be configured to facilitate control by surgeon 110-1 of other components of surgical system 100. For example, surgeon 110-1 may interact with user control system 104 to change a configuration or operating mode of surgical system 100, to change a display mode of surgical system 100, to generate additional control signals used to control surgical instruments attached to manipulator arms 112, to facilitate switching control from one robotic instrument to another, to facilitate interaction with other instruments and/or objects within the surgical space, or to perform any other suitable operation. To this end, user control system 104 may also include one or more input devices (e.g., foot pedals, buttons, switches, etc.) configured to receive input from surgeon 110-1.

Auxiliary system 106 may include one or more computing devices configured to perform primary processing operations of surgical system 100. The one or more computing devices included in auxiliary system 106 may control and/or coordinate operations performed by various other components (e.g., manipulating system 102 and/or user control system 104) of surgical system 100. For example, a computing device included in user control system 104 may transmit instructions to manipulating system 102 by way of the one or more computing devices included in auxiliary system 106. As another example, auxiliary system 106 may receive, from manipulating system 102, and process image data representative of images captured by an imaging device attached to one of manipulator arms 112.

In some examples, auxiliary system 106 may be configured to present visual content to surgical team members 110 who may not have access to the images provided to surgeon 110-1 at user control system 104. To this end, auxiliary system 106 may include a display monitor 114 configured to display one or more user interfaces, such as images (e.g., 2D images) of the surgical space, information associated with patient 108 and/or the surgical procedure, and/or any other visual content as may serve a particular implementation. For example, display monitor 114 may display images of the surgical space together with additional content (e.g., representations of target objects, graphical content, contextual information, etc.) concurrently displayed with the images. In some embodiments, display monitor 114 is implemented by a touchscreen display with which surgical team members 110 may interact (e.g., by way of touch gestures) to provide user input to surgical system 100.

Manipulating system 102, user control system 104, and auxiliary system 106 may be communicatively coupled one to another in any suitable manner. For example, as shown in FIG. 1, manipulating system 102, user control system 104, and auxiliary system 106 may be communicatively coupled by way of control lines 116, which may represent any wired or wireless communication link as may serve a particular implementation. To this end, manipulating system 102, user control system 104, and auxiliary system 106 may each include one or more wired or wireless communication interfaces, such as one or more local area network interfaces, Wi-Fi network interfaces, cellular interfaces, etc.

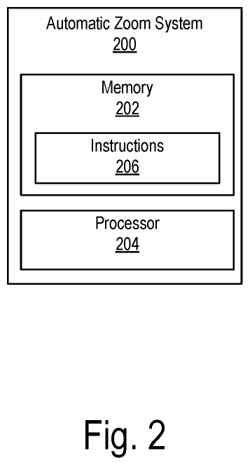

FIG. 2 shows an example automatic zoom system 200 that may be implemented according to principles described herein to implement a zoom feature associated with an imaging device. As shown in FIG. 2, automatic zoom system 200 (e.g., system 200) may include, without limitation, a memory 202 and a processor 204 selectively and communicatively coupled to one another. Memory 202 and processor 204 may each include or be implemented by hardware and/or software components (e.g., processors, memories, communication interfaces, instructions stored in memory for execution by the processors, etc.). In some examples, memory 202 and processor 204 may be implemented by a single device (e.g., a single computing device). In certain alternate examples memory 202 and processor 204 may be distributed between multiple devices and/or multiple locations as may serve a particular implementation.

Memory 202 may maintain (e.g., store) executable data used by processor 204 to perform any of the operations described herein. For example, memory 202 may store instructions 206 that may be executed by processor 204 to perform any of the operations described herein. Instructions 206 may be implemented by any suitable application, software, code, and/or other executable data instance.

Memory 202 may also maintain any data received, generated, managed, used, and/or transmitted by processor 204. For example, memory 202 may maintain any suitable data associated with implementing an automatic zoom feature. Such data may include, but is not limited to, data associated with depth map information associated with an imaging space, movement data (e.g., kinematics data for imaging devices and/or manipulator arms, etc.), object data, procedure data, image data (e.g., endoscopic images) of an imaging space, data defining guidance content associated with an image zoom feature, predefined threshold distance data, user interface content (e.g., graphical objects, notifications, etc.), and/or any other suitable data.

Processor 204 may be configured to perform (e.g., execute instructions 206 stored in memory 202) various processing operations associated with implementing a zoom feature associated with an imaging device. For example, processor 204 may automatically activate an image zoom feature to zoom in a view of an imaging space.

As used herein, the expression “automatically” means that an operation (e.g., an operation activating a zoom feature) or series of operations are performed without requiring further input from a user. For example, a determination that a user is moving or intends to move an imaging device closer to an object than a threshold distance may trigger automatic control of a computer-assisted surgical system to prevent the imaging device from moving closer to the object than the threshold distance and automatic activation of an image zoom feature to zoom in a view of the imaging space. These and other operations that may be performed by processor 204 are described herein.

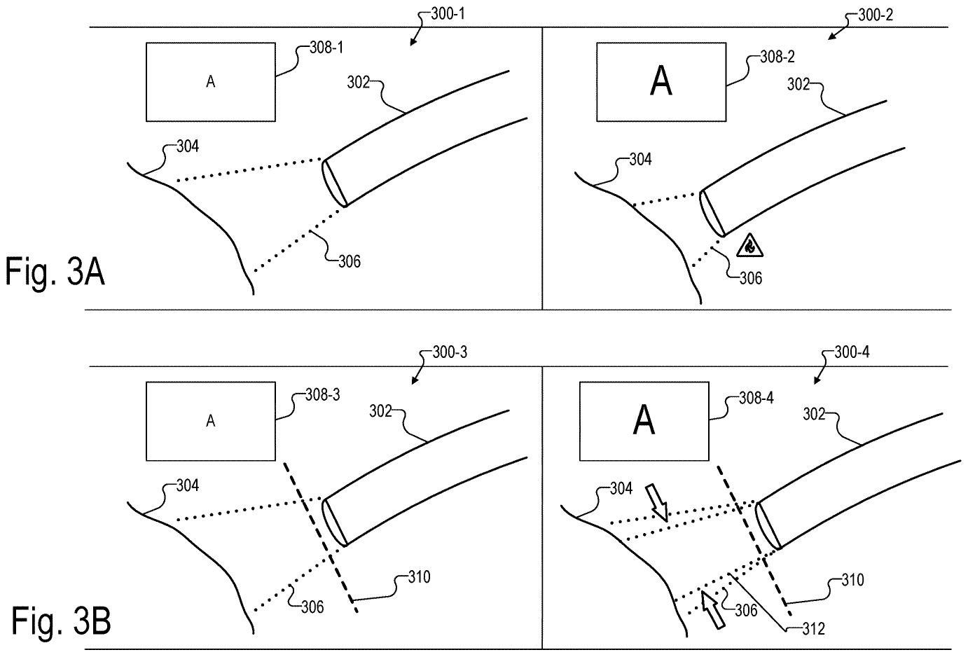

FIGS. 3A-3B illustrate example views 300 (e.g., views 300-1 through 300-4) of an imaging space that may be implemented according to principles described herein. As shown in view 300-1, an imaging device 302 is provided within the imaging space in relation to an object 304. Object 304 may represent any suitable type of object that may be located in an imaging space. For example, object 304 may represent a physical object such as anatomical tissue, an instrument, or any other suitable object that may be located in the imaging space. In certain examples, object 304 may represent an organ or a portion of an organ located in an imaging space. The distance between object 304 and imaging device 302 may be measured to the surface of object 304.

Imaging device 302 may correspond to any suitable type of imaging device that may be located within an imaging space. For example, imaging device 302 may correspond to an endoscope in certain examples. Imaging device 302 may capture one or more images in the imaging space. Any robotic instruments and/or object 304 that are within a field of view of imaging device 302 may be depicted in the image(s) captured by imaging device 302.

Imaging device 302 may include any suitable number or type of sensors as may serve a particular implementation. For example, imaging device 302 may include one or more cameras, depth sensors, capacitive sensors, ultrasonic sensors, optical time of flight (TOF) depth sensing sensors, and/or any other suitable type of sensor.

The sensors that may be included as part of imaging device 302 may be configured in any suitable manner and may be located on any suitable portion of imaging device 302. For example, imaging device 302 may include an image sensor at a distal end of imaging device 302. Additionally or alternatively, imaging device 302 may include an image sensor at a proximal end of imaging device 302. In such examples, light collected at the distal end of imaging device 302 may be transmitted to the image sensor at the proximal end by way of a light guide or in any other suitable manner.

Imaging device 302 may provide data representing visible light data of an imaging space. For example, imaging device 302 may capture visible light images of the imaging space that represent visible light sensed by imaging device 302. Visible light images may include images that use any suitable color and/or grayscale palette to represent a visible light-based view of the imaging space.

In certain additional or alternative examples, imaging device 302 may provide data representing non-visible light data. In such examples, imaging device 302 may include one or more infrared sensors and/or near infrared sensors.

Imaging device 302 may also provide data representing depth data of an imaging space or data that may be processed to derive depth data of the imaging space. For example, imaging device 302 may capture images of the imaging space that represent depth sensed by imaging device 302. Alternatively, imaging device 302 may capture images of the imaging space that may be processed to derive depth data of the imaging space. The depth information may be represented as depth images (e.g., depth map images obtained using a Z-buffer that indicates distance from imaging device 302 to each pixel point on an image of an imaging space), which may be configured to visually indicate depths of objects in the imaging space in any suitable way, such as by using different greyscale values to represent different depth values.

Images captured by imaging device 302 and/or derived from images captured by imaging device 302 (e.g., visible light images and depth images) may be used to facilitate detecting a position of imaging device 302 in relation to object 304 and/or determining when to automatically implement a zoom feature, such as described herein.

In certain examples, imaging device 302 may additionally or alternatively include any suitable other type of distance/proximity sensor(s). For example, imaging device 302 may include one or more capacitive sensors, ultrasonic sensors, optical time of flight (TOF) depth sensing sensors, and/or any other suitable type of sensor to determine the distance/proximity of imaging device 302 with respect to object 304.

In certain examples, imaging device 302 may include a plurality of lenses configured to facilitate an optical zoom function of imaging device 302. Imaging device 302 may include any suitable number and/or configuration of lenses as may serve a particular implementation. For example, imaging device 302 may include two lenses, three lenses, four lenses, and so forth.

As shown in FIG. 3A, imaging device 302 has a field of view 306 of object 304 at the distance that imaging device 302 is currently located from object 304 in view 300-1. Images 308 (e.g., images 308-1 through 308-4) represent images captured by imaging device 302 at the various positions depicted in FIGS. 3A and 3B. For example, image 308-1 in FIG. 3A shows an image captured by imaging device 302 at the position shown on the left side of FIG. 3A. An “A” is shown in images 308 for illustrative purposes only to show how images 308 of object 304 may change based on the distance imaging device 302 is from object 304 and/or the amount of zoom applied. It is understood that object 304 may correspond to tissue or some other object in the imaging space that does not include a letter “A” or any other letter on the surface thereof.

As shown in view 300-2, moving imaging device 302 relatively closer to object 304 increases the size of the “A” captured within image 308-2. However, moving imaging device 302 closer to object 304 may cause, for example, tissue burning and/or may result in obscuring an image captured by imaging device 302 due to fogging and/or splattering. In such examples, the tissue burning may be caused by illumination emitted at a distal end of imaging device 302.

In view of this, system 200 may implement one or more threshold distances that may be used in certain examples to determine when to either automatically activate or automatically deactivate an image zoom feature. In some implementations, a threshold distance may define a minimum safe distance that imaging device 302 (e.g., a distal end of imaging device 302) may be positioned with respect to an object that does not result in one or more of the above-described negative effects. The threshold distance may correspond to any suitable distance from an object in an imaging space as may serve a particular implementation. In the example shown in FIG. 3B, a threshold distance 310 is shown in dashed lines to illustrate a distance that a distal end of imaging device 302 may stay away from object 304 to effectively capture images of object 304 and avoid causing one or more of the above-described negative effects associated with moving imaging device 302 to close to object 304 (e.g., cause damage to object 304).

In certain examples, the threshold distance used by system 200 may be predefined. In certain alternative examples, system 200 may select a threshold distance based on one or more factors associated with the imaging space. System 200 may use any suitable factor to select a threshold distance as may serve a particular implementation. For example, the one or more factors may include a type of procedure performed in the imaging space, attributes of (e.g., a type or model of, a resolution of, an illuminator of, heat generated by, etc.) an imaging device used, and/or attributes of (e.g., a type of) an object imaged in the imaging space. For example, a first threshold distance may be selected for a first type of procedure, a second threshold distance may be selected for a second type of procedure, and a third threshold distance may be selected for a third type of procedure. The first, second, and third threshold distances may each be different from one another. Additionally or alternatively, the one or more factors may include taking into consideration the best possible distance to view small tissue details, the best possible distance to avoid a fusing problem due to too strong parallax/stereo impression, and/or any other suitable factor.

In certain examples, system 200 may facilitate a user selecting a threshold distance to use in a particular situation. In such examples, system 200 may provide any suitable user interface to facilitate the user selecting the threshold distance.

In certain examples, system 200 may be configured to dynamically change the threshold distance that is used during the course of a procedure performed in the imaging space. To illustrate, a first stage of the procedure may include capturing images of soft tissue where there is a relatively higher splatter risk. A second stage of the procedure may include capturing images of a bone of a subject where there is a relatively lower splatter risk. As such, it may be possible to move the imaging device closer to the bone of the subject than the soft tissue without resulting in splatter on the imaging device. Accordingly, system 200 may select a first threshold distance to use during the first stage of the procedure and a second threshold distance to use during the second stage of the procedure. The first threshold distance may be relatively longer than the second threshold distance. System 200 may be configured to automatically switch from using the first threshold distance to using the second threshold distance upon initiation of the second stage of the surgical procedure.

In certain examples, system 200 may be configured to implement any suitable object recognition algorithm to dynamically change the threshold distance that is used at a given time. For example, system 200 may use an object recognition algorithm to determine that imaging device 302 is currently capturing images of soft tissue. Based on such a determination, system 200 may select a first threshold distance to use while capturing images of the soft tissue. The view of imaging device 302 may then be adjusted and system 200 may determine that imaging device 302 is currently capturing images of bone or some other object that may not be as susceptible to burning and/or may not cause splatter on imaging device 302. As such, system 200 may select a second threshold distance to use while imaging device 302 captures images of the bone.

As shown in view 300-4 of FIG. 3B, instead of system 200 moving imaging device 302 closer to object 304 than threshold distance 310, system 200 may automatically activate an image zoom feature to narrow field of view 306 to field of view 312 and result in a relatively larger “A” in image 308-4. In so doing, the automatic zoom feature may provide an operator (e.g., surgeon 110-1) of imaging device 302 with an impression that imaging device 302 is moving closer to object 304 even though imaging device 302 does not actually move closer to object 304 than threshold distance 310.

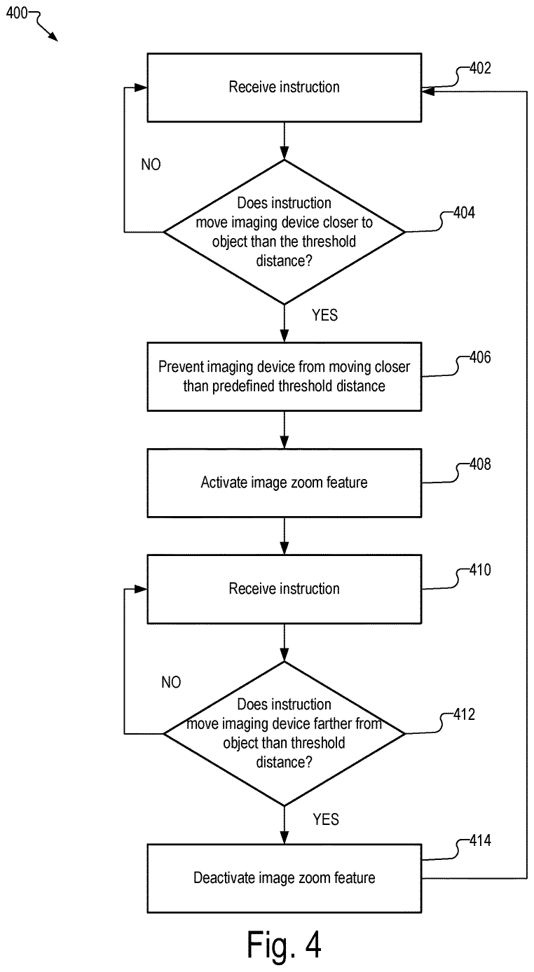

FIG. 4 illustrates a flow diagram 400 depicting various operations that may be performed by system 200 (e.g., processor 204) to automatically implement a zoom feature such as described herein. At operation 402, system 200 may receive an instruction to move an imaging device (e.g., imaging device 302) within an imaging space. The instruction may indicate that a user (e.g., surgeon 110-1) is moving or intends to move the imaging device with respect to an object in the imaging space.

System 200 may receive the instruction in any suitable manner. For example, the instruction may be received by way of surgeon 110-1 interacting with master controls of user control system 104 to direct a robotic manipulating arm (e.g., robotic manipulating arm 112-2) attached to the imaging device to move within the imaging space.

At operation 404, system 200 may determine, based on the instruction, whether the imaging device is located at a position that is at or near a threshold distance from an object. System 200 may determine whether the imaging device at or near the threshold distance in any suitable manner. For example, system 200 may access depth data that indicates how far the imaging device is from the object. Based on the depth data, system 200 may determine that the imaging device would be closer to the object than the threshold distance if the imaging device is moved to within the imaging space based on the instruction.

In certain examples, the determining that the imaging device is located at the position may include determining that a distal end of the imaging device is located at the position. In such examples, the distal end of the imaging device may include an illumination device that generates heat and may cause damage to the object if the distal end is moved closer to the object than the threshold distance.

In certain examples, system 200 may additionally or alternatively determine whether the imaging device is at or near the threshold distance by using an auto focus function of the imaging device. The auto focus function of the imaging device may indicate how far the imaging device is from the object at any given time.

If the answer at operation 404 is “NO,” the flow returns to operation 402. If the answer at operation 404 is “YES,” the flow proceeds to operation 406. At operation 406, system 200 may prevent the imaging device from moving closer to the object than the threshold distance. This may be accomplished in any suitable manner. For example, system 200 may instruct motors of the manipulator arm to which the imaging device is attached to stop moving towards the object once the threshold distance is reached or once the imaging device is within a predefined distance from the threshold distance. In addition, the motors may be prevented in any suitable manner from moving the imaging device in the z-direction past the threshold distance. However, the user may still be able to move the imaging device in the x-direction, the y-direction, and/or may be able to pivot the imaging device in any suitable manner.

In certain examples, the preventing of the imaging device from moving closer to the object than the threshold distance may include preventing the distal end of the imaging device from moving closer to the object than the threshold distance.

In certain examples, system 200 may be configured to provide feedback to inform a user of the computer-assisted surgical that the threshold distance has been reached. Such feedback may include any suitable feedback (e.g., any of visual, audible, and haptic feedback) and may be provided in any suitable manner. For example, system 200 may instruct a computer-assisted surgical system to cause one of the master controls of user control system 104 to vibrate to inform the user when the threshold distance is reached.

At operation 408, system 200 may automatically activate an image zoom feature associated with the imaging device to zoom in a view of the imaging space. With the image zoom feature activated, physical movement of the imaging device in the z-direction beyond the threshold distance and towards the object is replaced with zooming in on the object. This may give the impression to the user that the imaging device is moving closer to the object even though the imaging device does not physically move closer to the object than the threshold distance.

In certain examples, system 200 may obtain motion data associated with motion of the imaging device before automatically activating the image zoom feature.

Such motion data may include any suitable data associated with movement of the imaging device that may be used to control the image zoom function. For example, the motion data may be based on kinematic data, a motion sensor (e.g., one or more accelerometers) in the imaging device, data derived from images captured by the imaging device, and/or any other suitable data. System 200 may control the image zoom function in any suitable manner based on the motion data. For example, system 200 may determine a velocity of the imaging device while the image zoom function is active. System 200 may then control the zoom rate to mimic the effect of the imaging device moving at the same velocity.

In certain examples, the automatically activating of the image zoom feature may be imperceptible to a user. That is, the user may not be aware that the imaging device has physically stopped at or near the threshold distance and zoom is being applied. This may be accomplished in any suitable manner. For example, system 200 may determine a velocity of a distal tip of the imaging device. Based on the determined velocity, system 200 may determine a rate of change of zoom applied by the zoom feature to substantially match the perceptible output of moving the distal tip of the imaging device closer to the object at the determined velocity.

In certain examples, the view of the imaging space may include a view, from a virtual camera, of a 3D model of at least a portion of the imaging space. Such a 3D model may be generated in any suitable manner. For example, system 200 may generate the 3D model in real time or near real time based on images captured by the imaging device. In examples where system 200 uses a real time or near real time 3D model (e.g., a simultaneous localization and mapping (SLAM) based 3D model) of the imaging space, system 200 may determine a 3D trajectory of the imaging device. Once the threshold distance is reached, system 200 may prevent the imaging device from moving closer to the object than the threshold distance by stopping the imaging device at a point along the 3D trajectory. The automatically activating of the zoom feature may include moving the virtual camera closer to the object than the imaging device. For example, system 200 may move the virtual camera along that 3D trajectory (and at a similar velocity) past the point after stopping the imaging device at the point. In such examples, the virtual camera may produce a virtual image that mimics moving the imaging device closer to the object in a manner that is imperceptible to the user.

In certain alternative implementations, system 200 may be configured to provide a notification to a user that the image zoom feature has been activated. Such a notification may be provided in any suitable manner. For example, system 200 may provide an audio notification (e.g., a voice message saying “zoom feature activated”), a text notification (e.g., the text “zoom feature activated”), an augmented reality notification, an icon notification (e.g., a magnifying glass icon), and/or any other suitable notification.

System 200 may automatically activate the image zoom feature in any suitable manner. In certain examples, the automatically activating of the image zoom feature may include activating an optical zoom feature of the imaging device. In such examples, the imaging device may be configured with a plurality of lenses that may be configured to facilitate optically zooming in on an object in any suitable manner. For example, system 200 may be configured to change focal lengths between lenses included in the plurality of lenses to narrow the field of view and zoom in a view of the imaging space.

In certain examples, the automatically activating of the image zoom feature may include activating a digital zoom feature of the imaging device. System 200 may implement a digital zoom feature in any suitable manner. For example, system 200 may decrease the field of view of an image by cropping the image captured by the imaging device down to an area with the same aspect ratio of the original image and scaling the cropped image up to the dimensions of the original image. In such examples, the image zoom feature may include system 200 digitally zooming in on a particular portion of an image captured by the imaging device.

In certain examples, with a digital zoom feature, system 200 may be configured to select which portion of a captured image to zoom in on based on an input provided by a user. System 200 may be configured to detect any suitable user input as may serve a particular implementation. For example, system 200 may be configured to track the gaze of one or more eyes of a user of a computer-assisted surgical system and may digitally zoom in on a portion of the image that the user is currently looking at. In such examples, system 200 may be configured to implement any suitable gaze tracking methodology as may serve a particular implementation. Additionally or alternatively, system 200 may select which portion of a captured image to zoom in on based on a trajectory of the imaging device. Additionally or alternatively, system 200 may select which portion of a captured image to zoom in on based on a detected portion of interest in an image captured by the imaging device In certain alternative examples, the automatically activating of the image zoom feature may include activating both an optical zoom feature and a digital zoom feature. In such examples, system 200 may first optically zoom in on the object and may then digitally zoom in on any suitable portion of the image captured by the imaging device.

While the zoom feature is active, system 200 may dynamically adjust the amount of zoom provided at any given time based on instructions to move the imaging device in the z-direction, the x-direction, the y-direction, and/or based on pivoting movements that change the viewing direction of the imaging device.

In addition, while the image zoom feature is active, system 200 may perform any suitable processing operation(s) on images captured by the imaging device to give the impression that the imaging device is closer to the object than the threshold distance. For example, system 200 may crop an image captured by the imaging device, dynamically adjust a perspective of the image, dynamically adjust distortion of all or part of the image, apply three-dimensional (“3D”) texture mapping to the image, dynamically adjust the scale of a 3D model or 3D rendering that may be displayed together with the image, artificially brighten the image, and/or dynamically adjust a zoom ratio depending on how close the user would move the imaging device to the object.

In certain examples, system 200 may determine a virtual zoomed in pivot point for the imaging device. Such a virtual zoomed in pivot point may be determined in any suitable manner. For example, a virtual zoomed in pivot point may be determined based on the position of the imaging device at or near the threshold distance. Any change in the physical position and/or orientation of the imaging device also changes the virtual zoomed in pivot point. Further, the movement of the virtual zoomed in pivot point while the image zoom feature is active may change and/or otherwise affect the kinematics of the manipulator arm attached to the imaging device. Accordingly, system 200 may take into consideration the virtual zoomed in pivot point when zooming in and/or processing a zoomed in image. In addition, system 200 may take into consideration the virtual zoomed in pivot point when the imaging device is physically moved and/or pivoted while the image zoom feature is activated. For example, when a user input is provided to move the imaging device in the x-direction and/or the y-direction, system 200 may take into consideration the virtual zoomed in pivot point, such as by using the virtual zoomed in pivot point as an input when translating the user input into a movement and/or by applying the movement based on the virtual zoomed in pivot point. Similarly, when a user input is additionally or alternatively provided to rotate the imaging device along a yaw and/or pitch axis while the image zoom feature is active, system 200 may consider and use the virtual zoomed in pivot point as an input when translating the user input into a rotating motion along the yaw and/or pitch axis. For example, the amount of yaw/pitch rotation needed at the virtual zoomed in pivot point may be greater than that needed at the physical pivot point where the imaging device is physically located. Similarly, when a user input is additionally or alternatively provided to pivot the imaging device while the image zoom feature is active, system 200 may consider and use the virtual zoomed in pivot point as an input when translating the user input into a pivoting motion and/or by applying the pivot based on the virtual zoomed in pivot point. This may allow the virtual location of the virtual pivot point to change within the imaging space even though the physical position of the imaging device may not change and/or may allow actual movement of the imaging device to be based on a zoomed in viewpoint such that the virtual zoomed in viewpoint remains realistic to a user.

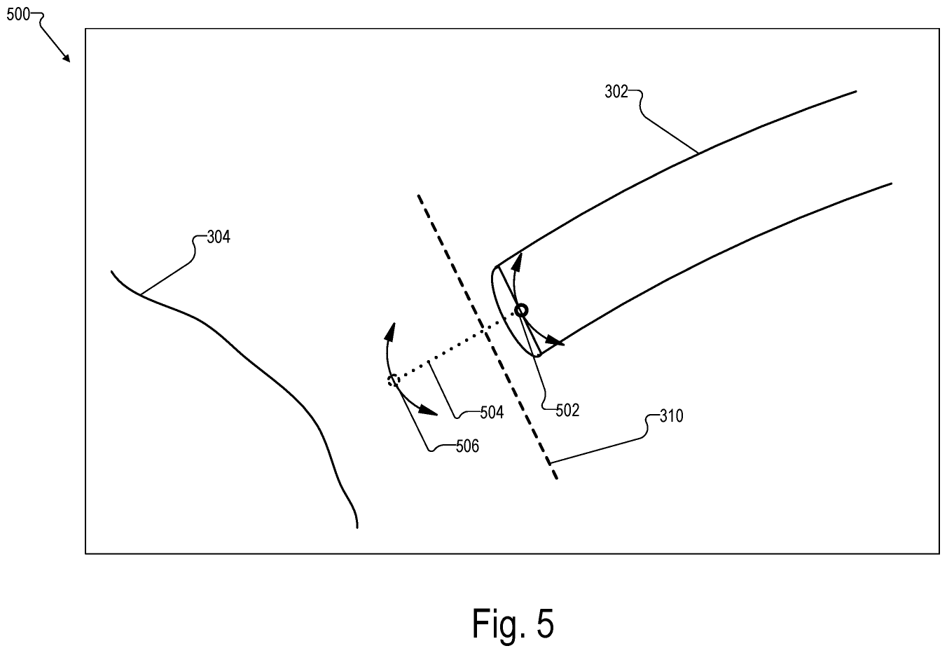

FIG. 5 shows an example of a view 500 of an imaging space while an image zoom feature is active. As shown in FIG. 5, imaging device 302 is prevented from moving closer to object 304 than threshold distance 310. A pivot point 502 corresponds to an actual pivot point of imaging device 302. Imaging device 302 is currently zoomed in by an amount associated with dotted line 504. A virtual zoomed in pivot point 506 corresponds to a virtual pivot point of imaging device 302 based on the amount of zoom associated with dotted line 504. System 200 may be configured to dynamically adjust the location of virtual zoomed in pivot point 506 and/or image processing associated with virtual zoomed in pivot point 506 in any suitable manner based on movement of imaging device 302 in relation to object 304. For example, system 200 may adjust the viewing angle associated with virtual zoomed in pivot point 506 based on imaging device pivoting in the direction of the arrows shown in FIG. 5 with respect to pivot point 502.

In certain examples, while the image zoom feature is active, system 200 may calculate a virtual camera calibration (e.g., calibration of the focal length) from the perspective of the virtual zoomed in pivot point. System 200 may implement such a virtual camera calibration in any suitable manner to facilitate providing a zoomed in image to a user. For example, system 200 may adjust the depth mapping of the imaging space, the rendering of user interface elements, and/or any other aspect based on the virtual camera calibration.

System 200 may display a zoomed in image to a user of a computer-assisted surgical system in any suitable manner. For example, the zoomed in image may be displayed to surgeon 110-1 by way of a stereoscopic image viewer of user control system 104. In certain examples, system 100 may display the zoomed in image together with an image captured by the imaging device that is not zoomed in. This may be accomplished in any suitable manner. For example, system 200 may display a first window that depicts the zoomed in image and a second relatively smaller window that depicts an image that is not zoomed in. In certain examples, the second relatively smaller window may be overlaid over the first window (e.g., at a corner of the first window).

At operation 410, system 200 may receive an additional instruction to move the imaging device to an additional position within the imaging space.

At operation 412, system 200 may determine whether the additional instruction will move the imaging device farther from the object than the threshold distance. If the answer at operation 412 is “NO,” the flow returns to operation 410. If the answer at operation 412 is “YES,” the flow proceeds to operation 414 in which the image zoom feature is automatically deactivated. System 200 may automatically deactivate the image zoom feature in any suitable manner. For example, based on the instruction, system 200 may automatically zoom out until no more zoom is implemented by system 200. After zooming out, system 200 may deactivate the image zoom feature at operation 414 when no more zoom is applied and/or when the imaging device begins moving toward the additional position associated with the additional instruction at operation 410.

In certain examples, system 200 may implement a different threshold distance for deactivating an image zoom feature than is used for activating an image zoom feature. For example, system 200 may use a first threshold distance to determine when to activate the image zoom feature and may use a second threshold distance, different than the first threshold distance, to determine when to deactivate the image zoom feature. In certain examples, the second threshold distance may be greater than the first threshold distance. Having the second threshold distance be greater than the first threshold distance may improve usability by preventing repeatedly and quickly switching back and forth between modes based on a single threshold boundary.

After operation 414, the flow may then return to operation 402 in which system 200 waits to receive an additional instruction associated with the movement of the imaging device in the imaging space.

System 200 may repeat operations 402-414 any suitable number of times during the course of a procedure performed in an imaging space to facilitate automatically activating and automatically deactivating an image zoom feature associated with an imaging device.

In certain examples, system 200 may provide an option for a user to manually deactivate the image zoom feature of an imaging device. Such an option may provide the user with full control of the imaging device also at close distances from an object in an imaging space.

In certain alternative examples, system 200 may automatically activate an image zoom feature based on a detected attribute of an object in an imaging space. System 200 may use any suitable attribute of an object to determine when to automatically activate an image zoom feature. For example, system 200 may use the temperature of an object such as tissue as an attribute that may be used to determine when to automatically activate an image zoom feature in certain implementations. In such examples, system 200 may detect, in any suitable manner, a surface temperature of the tissue that is being imaged by an imaging device. System 200 may determine whether the temperature is above a predefined threshold that may cause damage to the tissue. If the temperature is above the predefined threshold temperature, system 200 may automatically activate the image zoom feature and automatically move the imaging device any suitable distance away from the tissue to thereby reduce the surface temperature. While the imaging device moves away from the tissue, system 200 may gradually increase the zoom, which may give the impression to a user that the imaging device is not moving away from the tissue. In so doing, system 200 may prevent or mitigate damage that may occur to the tissue due to the heat emitted from the imaging device while still providing an up-close field of view that may be desired by a user.

Although the preceding disclosure describes an automatic zoom feature that automatically zooms in a view of an imaging space, it is understood that concepts such as those described herein may also be applied in the opposite direction. For example, an automatic zoom feature may additionally or alternatively include automatically zooming out based on a condition being satisfied. For example, if a user would like a wider field of view, the user may move an imaging device away from an object in the imaging space. However, at some point, the imaging device may not be able to physically move farther backward (e.g., without exiting a body cavity and/or due to kinematic constraints). In such examples, system 200 may determine in any suitable manner that the imaging device is approaching an additional threshold distance away from an object, may prevent the imaging device from moving past the additional threshold distance, and may automatically zoom out (e.g., digitally and/or optically) to widen the field of view without physically moving the imaging device past the additional threshold distance.

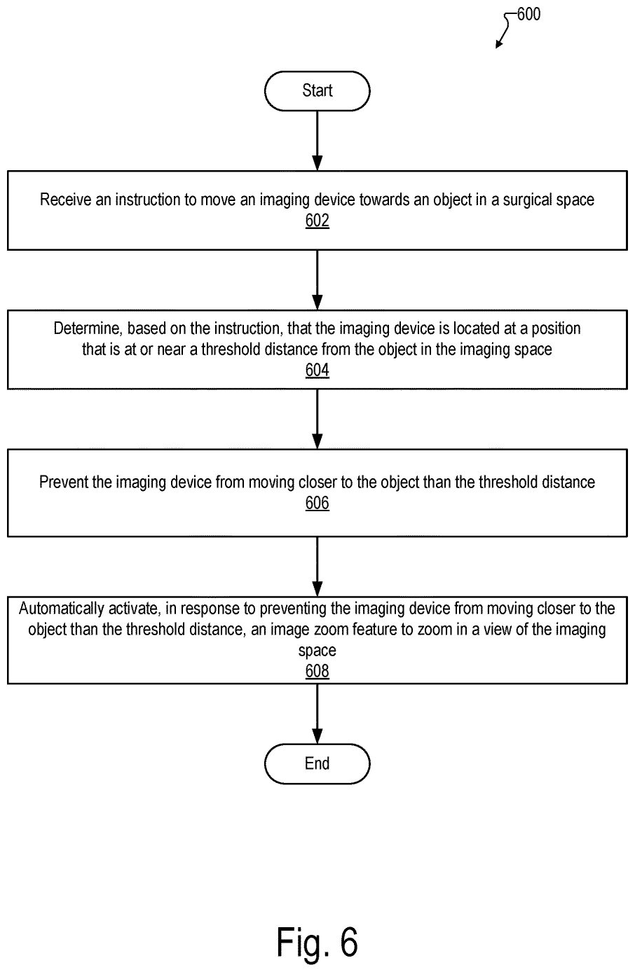

FIG. 6 illustrates an example method 600 for implementing a zoom feature associated with an imaging device in an imaging space. While FIG. 6 illustrates example operations according to one embodiment, other embodiments may omit, add to, reorder, and/or modify any of the operations shown in FIG. 6. One or more of the operations shown in FIG. 6 may be performed by a system such as system 200, any components included therein, and/or any implementation thereof.

At operation 602, an automatic zoom system (e.g., automatic zoom system 200) may receive an instruction to move an imaging device, which is located within an imaging space and coupled to a robotic manipulating arm of a computer-assisted surgical system, towards an object in the imaging space. Operation 602 may be performed in any of the ways described herein.

At operation 604, the automatic zoom system may determine, based on the instruction, that the imaging device is located at a position that is at or near a predefined distance from the object in the imaging space. Operation 604 may be performed in any of the ways described herein.

At operation 606, the automatic zoom system may prevent the imaging device from moving closer to the object than the threshold distance. Operation 606 may be performed in any of the ways described herein.

At operation 608, the automatic zoom system may automatically activate, in response to the imaging device being prevented from moving closer to the object than the threshold distance, an image zoom feature to zoom in a view of the imaging space. Operation 608 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Illustrative non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Illustrative volatile storage media include, but are not limited to, RAM (e.g., dynamic RAM).

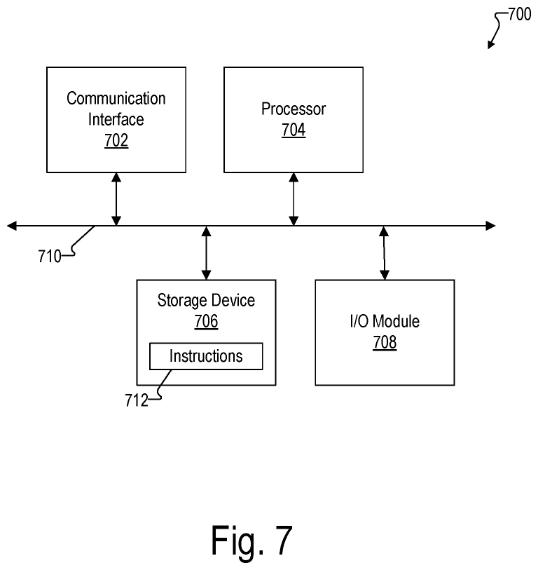

FIG. 7 illustrates an example computing device 700 that may be specifically configured to perform one or more of the processes described herein. As shown in FIG. 7, computing device 700 may include a communication interface 702, a processor 704, a storage device 706, and an input/output (“I/O”) module 708 communicatively connected one to another via a communication infrastructure 710. While an example computing device 700 is shown in FIG. 7, the components illustrated in FIG. 7 are not intended to be limiting. Additional or alternative components may be used in other embodiments. Components of computing device 700 shown in FIG. 7 will now be described in additional detail.

Communication interface 702 may be configured to communicate with one or more computing devices. Examples of communication interface 702 include, without limitation, a wired network interface (such as a network interface card), a wireless network interface (such as a wireless network interface card), a modem, an audio/video connection, and any other suitable interface.

Processor 704 generally represents any type or form of processing unit capable of processing data and/or interpreting, executing, and/or directing execution of one or more of the instructions, processes, and/or operations described herein. Processor 704 may perform operations by executing computer-executable instructions 712 (e.g., an application, software, code, and/or other executable data instance) stored in storage device 706.

Storage device 706 may include one or more data storage media, devices, or configurations and may employ any type, form, and combination of data storage media and/or device. For example, storage device 706 may include, but is not limited to, any combination of the non-volatile media and/or volatile media described herein. Electronic data, including data described herein, may be temporarily and/or permanently stored in storage device 706. For example, data representative of computer-executable instructions 712 configured to direct processor 704 to perform any of the operations described herein may be stored within storage device 706. In some examples, data may be arranged in one or more databases residing within storage device 706.

I/O module 708 may include one or more I/O modules configured to receive user input and provide user output. One or more I/O modules may be used to receive input for a single virtual experience. I/O module 708 may include any hardware, firmware, software, or combination thereof supportive of input and output capabilities. For example, I/O module 708 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 708 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 708 is configured to provide graphical data to a display for presentation to a user. The graphical data may be representative of one or more graphical user interfaces and/or any other graphical content as may serve a particular implementation.

In some examples, any of the systems, computing devices, and/or other components described herein may be implemented by computing device 700. For example, memory 202 may be implemented by storage device 706, and processor 204 may be implemented by processor 704.

In the preceding description, various example embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
one or more processors communicatively coupled to the memory and configured to execute the instructions to perform a process comprising:
receiving an instruction to move an imaging device, which is located within an imaging space and coupled to a robotic manipulating arm of a computer-assisted surgical system, towards an object in the imaging space;
determining, based on the instruction, that the imaging device is located at a position that is at or near a threshold distance from the object in the imaging space;
preventing the imaging device from moving closer to the object than the threshold distance; and
automatically activating, in response to preventing the imaging device from moving closer to the object than the threshold distance, an image zoom feature to zoom in a view of the imaging space.

2. The system of claim 1, wherein:
the determining that the imaging device is located at the position includes determining that a distal end of the imaging device is located at the position; and
the preventing of the imaging device from moving closer to the object than the threshold distance includes preventing the distal end of the imaging device from moving closer to the object than the threshold distance.

3. The system of claim 1, wherein the process further comprises:
receiving an additional instruction to move the imaging device to an additional position within the imaging space;
determining, based on the additional instruction, that the additional position is farther than the threshold distance from the object; and
automatically deactivating the image zoom feature when the imaging device begins moving toward the additional position.

4. The system of claim 1, wherein the process further comprises selecting the threshold distance based on one or more factors associated with the imaging space.

5. The system of claim 4, wherein the one or more factors include at least one of a type of procedure performed in the imaging space, a type of imaging device, or a type of object imaged in the imaging space.

6. The system of claim 1, wherein the automatically activating of the image zoom feature is imperceptible to a user of the computer-assisted surgical system.

7. The system of claim 1, further including providing a notification to a user of the computer-assisted surgical system that the image zoom feature is active.

8. The system of claim 1, wherein the automatically activating of the image zoom feature includes activating an optical zoom feature of the imaging device.

9. The system of claim 1, wherein the automatically activating of the image zoom feature includes activating a digital zoom feature.

10. The system of claim 1, wherein the automatically activating of the image zoom feature includes activating both an optical zoom feature and a digital zoom feature.

11. The system of claim 1, wherein the automatically activating of the image zoom feature to zoom in a view of the imaging space includes zooming in on a portion of an image captured by the imaging device.

12. The system of claim 11, wherein the portion of the image captured by the imaging device is selected based on at least one of eye gaze tracking of one or more eyes of a user of the computer-assisted surgical system, a trajectory of the imaging device, or a detected portion of interest in an image captured by the imaging device.

13. The system of claim 1, wherein the determining that the imaging device is at the position that is at or near the threshold distance from the object in the imaging space includes using a depth map of the imaging space.

14. The system of claim 1, wherein the determining that the imaging device is at the position that is at or near the threshold distance from the object in the imaging space includes using an auto focus function of the imaging device.

15. The system of claim 1, wherein the preventing of the imaging device from moving closer to the object than the threshold distance includes providing haptic feedback to a user of the computer-assisted surgical system.

16. The system of claim 1, wherein the view of the imaging space includes a view, from a virtual camera, of a three-dimensional (3D) model of at least a portion of the the imaging space.

17-18. (canceled)

19. The system of claim 16, wherein:
the instruction to move the imaging device includes an instruction to move the imaging device along a 3D trajectory towards the object in the imaging space;
preventing the imaging device from moving closer to the object than the threshold distance includes stopping the imaging device at a point along the 3D trajectory; and
automatically activating the image zoom feature includes moving the virtual camera along the 3D trajectory past the point after stopping the imaging device at the point.

20. (canceled)

21. The system of claim 1, wherein the process further comprises determining a virtual zoomed in pivot point for the imaging device based on the position of the imaging device.

22. A computer program product embodied in a non-transitory computer readable storage medium and comprising computer instructions for performing a process comprising:
receiving an instruction to move an imaging device, which is located within an imaging space and coupled to a robotic manipulating arm of a computer-assisted surgical system, towards an object in the imaging space;
determining, based on the instruction, that the imaging device is located at a position that is at or near a threshold distance from the object in the imaging space;
preventing the imaging device from moving closer to the object than the threshold distance; and
automatically activating, in response to preventing the imaging device from moving closer to the object than the threshold distance, an image zoom feature to zoom in a view of the imaging space.

23-24. (canceled)

25. A method comprising:
receiving, by an automatic zoom system, an instruction to move an imaging device, which is located within an imaging space and coupled to a robotic manipulating arm of a computer-assisted surgical system, towards an object in the imaging space;
determining, by the automatic zoom system and based on the instruction, that the imaging device is at or near a threshold distance from the object in the imaging space;
preventing, by the automatic zoom system, the imaging device from moving closer to the object than the threshold distance; and
automatically activating, by the automatic zoom system and in response to preventing the imaging device from moving closer to the object than the threshold distance, an image zoom feature to zoom in a view of the imaging space.

26-27. (canceled)
